(19) United States
(12) Patent Application Publication
(10) Pub. No.: US 2016/0339298 A1
(43) Pub. Date: Nov. 24, 2016

(54) Exercise Incentive System

(71) Applicant: Gennadiy Kats, Marina Del Rey, CA (US)

(72) Inventor: Gennadiy Kats, Marina Del Rey, CA (US)

(21) Appl. No.: 15/161,435

(22) Filed: May 23, 2016

Related U.S. Application Data
(60) Provisional application No. 62/165,940, filed on May 23, 2015.

(51) Int. Cl. A63B 24/00; A61B 5/00; G06Q 30/02; A61B 5/0205

(52) U.S. Cl. CPC A63B 24/0059; A63B 24/0062; A61B 5/0205; A61B 5/486; A61B 5/4866; G06Q 30/0236; G06Q 30/0201; A61B 5/024

(57) ABSTRACT

An exercise incentive system receives exercise sensor data originating from at least one exercise tracking device. The at least one exercise tracking device transmits exercise sensor data determined from at least one exercise sensor. The exercise incentive system estimates a personalized caloric baseline for an exerciser. At least a portion of incremental calories burned by the exerciser over at least one period of time are determined and converted into at least one exercise credit.

### CROSS-REFERENCE TO RELATED APPLICATIONS

This application claims the benefit of U.S. Provisional Application No. 62/165,940, filed May 23, 2015, which is hereby incorporated by reference in its entirety.

### BACKGROUND

Targeted advertising is often based on limited demographic information. Consumers in general are often reluctant to share personal information. However, some consumers may be willing to exchange personal information for meaningful incentives. What is needed is an exercise to credit conversion system configured to exchange exerciser demographic information for exercise credit and/or exerciser targeted advertisement opportunities.

Various programs exist that attempt to incentivize exercisers to exercise. Many of these programs rely on estimations of daily calories burned. Such programs may be based at least in part on daily estimates for sedentary and/or resting calories burned by an exerciser. Such programs may be exploited by exercisers in a variety of ways. What is needed is an exercise incentive system based at least in part on the incremental calories burned during exercise.

### BRIEF DESCRIPTION OF THE SEVERAL VIEWS OF THE DRAWINGS

FIG. 1 is an example block diagram showing elements of an exercise to credit conversion network as per an aspect of various embodiments.

FIG. 2 is an example flow diagram of exercise to credit conversion as per an aspect of an embodiment.

FIG. 3 is an example block diagram showing elements of an exercise incentive network as per an aspect of various embodiments.

FIG. 4 is an example flow diagram of incremental calorie conversion as per an aspect of an embodiment.

FIG. 5 is an example flow diagram of providing exerciser demographic information as per aspects of various embodiments.

FIG. 6 is a block diagram of a computing environment in which aspects of embodiments of the present invention may be practiced.

### DETAILED DESCRIPTION OF EMBODIMENTS

At least some embodiments of the present invention convert exercise data into at least one exercise credit. At least some embodiments of the present invention present incentives to exercise.

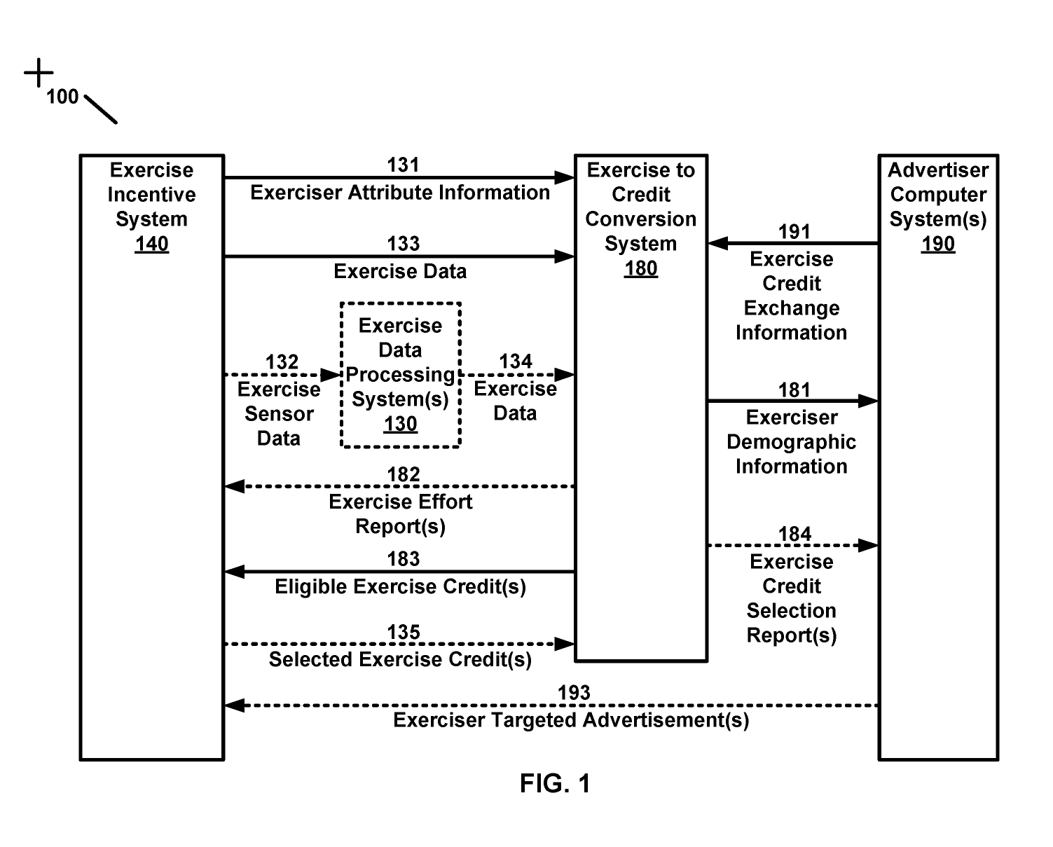

FIG. 1 is an example block diagram showing elements of an exercise to credit conversion network 100 comprising an exercise incentive system 140, an exercise to credit conversion system 180, and at least one advertiser computer system 190. At least one of the exercise incentive system 140, the exercise to credit conversion system 180, and the at least one advertiser computer system 190 may be coupled to a global computer network. The exercise to credit conversion system 180 may be configured to access at least one storage device. The at least one storage device may be coupled to the global computer network. The exercise to credit conversion system 180 may be programmed to perform a method of converting exercise data (e.g. 133 or 134) into at least one exercise credit. The method performed by the exercise to credit conversion system 180 may comprise receiving exercise to credit exchange information 191 from at least one advertiser computer system 190. The exercise to credit exchange information 191 may comprise at least one exchange rate, at least one exchange maximum, at least one exchange qualifier, exchange terms, combinations thereof, and/or the like. The at least one exchange rate may, for example, comprise incremental calories, steps, exercises, dollars, cents, a percent discount, combinations thereof, and/or the like. The at least one exchange maximum may, for example, comprise a maximum amount of dollars, a maximum amount of percent discount, a maximum amount of exercise credit per day, a maximum amount of calories accepted per day, a maximum amount of incremental calories accepted per day, a maximum amount of exercise credit per advertiser, combinations thereof, and/or the like. The at least one exchange qualifier may, for example, comprise a minimum purchase amount, a maximum amount of exercise credit per purchase, a maximum discount per purchase, combinations thereof, and/or the like. The exchange terms may, for example, comprise a blackout date, at least one product, at least one product type, an expiration date, combinations thereof, and/or the like. According to some of the various embodiments, the exercise incentive system 140 may be managed by a first party. The exercise to credit conversion system 180 may be managed by a second party. The at least one advertiser computer system 190 may be managed by at least one third party.

According to some of the various embodiments, a method performed by an exercise to credit conversion system 180 may comprise receiving exerciser attribute information 131 from an exercise incentive system 140. The exerciser attribute information 131 may comprise age, height, weight, gender, combinations thereof, and/or the like. The exerciser attribute information 131 may comprise name, address, birthday, race, marital status, employment status, income, age of at least one family member, combinations thereof, and/or the like. The exerciser attribute information 131 may comprise activity tracker information. The activity tracker information may comprise at least one activity tracker brand, at least one activity tracker model, at least one user account associated with the at least one activity tracker brand, combinations thereof, and/or the like. The exerciser attribute information 131 may comprise at least one fitness goal. The at least one fitness goal may be based at least in part on calories, steps, miles, time, heart rate, power output, combinations thereof, and or the like. The exerciser attribute information 131 may comprise at least one exercise preference. The at least one exercise preference may comprise an exercise type, an exercise duration, a daily goal, a weekly goal, at least one exercise location, combinations thereof, and/or the like. The exerciser attribute information 131 may comprise at least one shopping preference. The at least one shopping preference may comprise at least one product, at least one product category, at least one merchant, at least one priority, at least one location, combinations thereof, and/or the like. The exerciser attribute information 131 may comprise at least one location. The at least one location may be associated with the exercise incentive system 140. The at least one location may be supplied by an exerciser employing the exercise incentive system 140.

According to some of the various embodiments, a method performed by an exercise to credit conversion system 180 may comprise receiving exercise data (e.g. 133 or 134) from an exercise incentive system 140 and/or from at least one exercise data processing system 130. Exercise data (e.g. 134) received from the at least one exercise data processing system 130 may be associated with an exerciser employing the exercise incentive system 140. Exercise data (e.g. 134) received from the at least one exercise data processing system 130 may be associated with an activity tracker employed by the exerciser employing the exercise incentive system 140. At least some of the exercise data (e.g. 133 or 134) may be derived from exercise sensor data 132. The exercise sensor data 132 may be electronically communicated from a fitness tracker, a biometric monitoring device, a heart rate monitor, a heart pulse transmitter, a position tracker, a movement tracker, combinations thereof, and/or the like. The fitness tracker, biometric monitoring device, heart rate monitor, heart pulse transmitter, position tracker, movement tracker, combinations thereof, and/or the like may be embedded in exercise incentive system 140. The method performed by an exercise to credit conversion system 180 may comprise storing the exercise data as stored exercise data in at least one of at least one storage device. The stored exercise data may comprise incremental calories burned, exercise calories, total calories burned, incremental steps, exercise steps, total steps, combinations thereof, and/or the like.

According to some of the various embodiments, a method performed by an exercise to credit conversion system180 may comprise creating exerciser demographic information 181. The exerciser demographic information 181 may be based, at least in part, on exerciser attribute information 131 and/or stored exercise data. The exerciser demographic information 181 may comprise a fitness level for an exerciser. The fitness level for an exerciser may be based, at least in part, on the stored exercise data. The stored exercise data may be associated with the exerciser. The stored exercise data may comprise data associated with a plurality of exercises. The method performed by an exercise to credit conversion system 180 may comprise communicating the exerciser demographic information 181 to at least one advertiser computer system 190. At least one of the at least one advertiser computer system 190 may be configured to communicate at least one exerciser targeted advertisement 193 to an exercise incentive system 140. The at least one exerciser targeted advertisement 193 may be based, at least in part, on the exerciser demographic information 181.

According to some of the various embodiments, a method performed by an exercise to credit conversion system 180 may comprise creating at least one eligible exercise credit 183. The at least one eligible exercise credit 183 may be based, at least in part, on exercise to credit exchange information 191 and/or stored exercise data. The exercise to credit exchange information may comprise information received from a plurality of advertiser computer systems 190. The stored exercise data may comprise data created from a plurality of exercises. The stored exercise data may comprise data created from a plurality of exercises across a plurality of days, weeks, and/or months. The at least one eligible exercise credit 183 may comprise a digital currency, a currency, at least one electronic coupon, at least one credit card code, at least one debit card code, at least one gift card code, combinations thereof, and/or the like. The method performed by an exercise to credit conversion system 180 may comprise communicating the at least one eligible exercise credit 183 to an exercise incentive system 140. The method performed by an exercise to credit conversion system 180 may comprise receiving a selection of at least one of the at least one eligible exercise credit 135 communicated from the exercise incentive system 140.

According to some of the various embodiments, a method performed by an exercise to credit conversion system 180 may comprise creating at least one exercise credit selection report 184. The at least one exercise credit selection report 184 may be based, at least in part, on at least one selection of at least one of at least one eligible exercise credit 135 from an exercise incentive system 140. The method performed by an exercise to credit conversion system 180 may comprise communicating the at least one exercise credit selection report 184 to at least one of at least one advertiser computer system 190.

According to some of the various embodiments, a method performed by an exercise to credit conversion system 180 may comprise communicating at least one exercise effort report 182 to an exercise incentive system 140. The at least one exercise effort report 182 may comprise a baseline, at least one exercise, at least one eligible exercise credit 183, incremental calories burned, exercise calories, total calories burned, incremental steps, exercise steps, total steps, a fitness level, combinations thereof, and/or the like. The at least one exercise effort report 182 may be based, at least in part, on a plurality of exercises. The incremental calories burned, exercise calories, total calories burned, incremental steps, exercise steps, total steps, combinations thereof, and/or the like may be determined for each of the plurality of exercises. The at least one exercise effort report 182 may be based, at least in part, on stored exercise data.

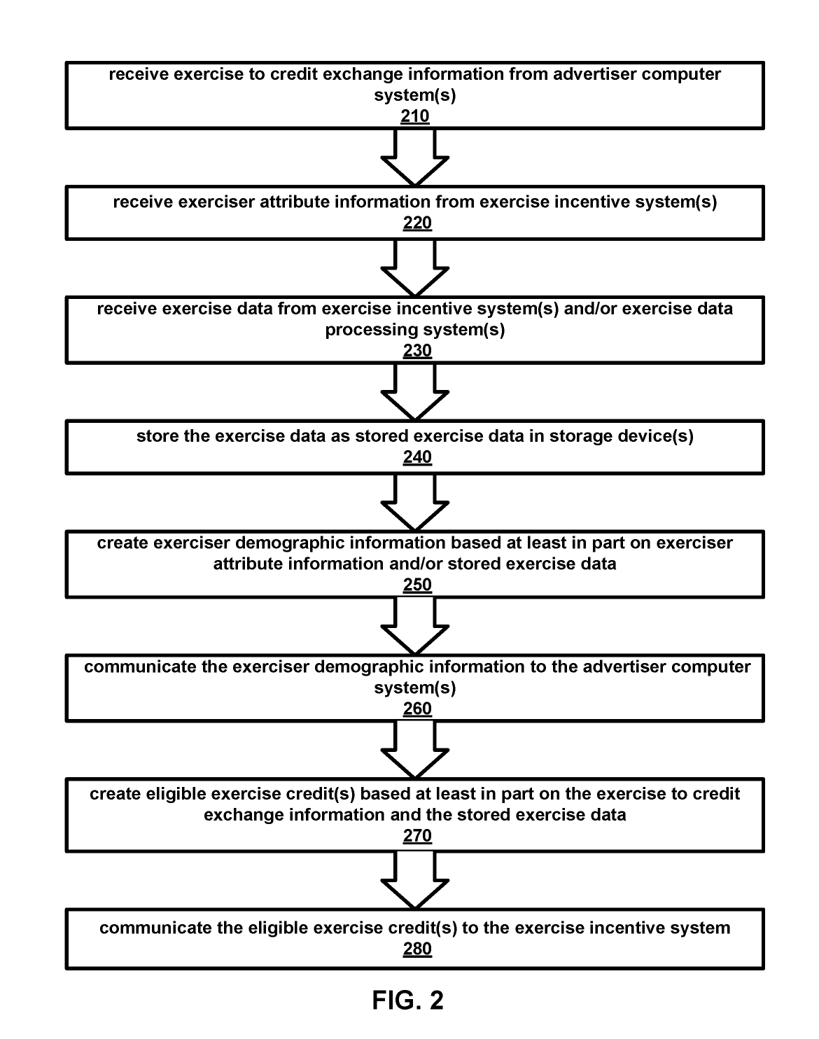

FIG. 2 is an example flow diagram of exercise to credit conversion as per an aspect of an embodiment. Exercise to credit exchange information may be received from at least one advertiser computer system at 210. Exerciser attribute information may be received from at least one exercise incentive system at 220. Exercise data may be received from at least one exercise incentive system and/or exercise data processing system at 230. Exercise data may be stored as stored exercise data in at least one storage device at 240. Exerciser demographic information may be created based at least in part on exerciser attribute information and/or stored exercise data at 250. Exerciser demographic information may be communicated to at least one advertiser computer system at 260. At least one eligible exercise credit may be created based at least in part on exercise to credit exchange information and stored exercise data at 270. At least one eligible exercise credit may be communicated to an exercise incentive system at 280.

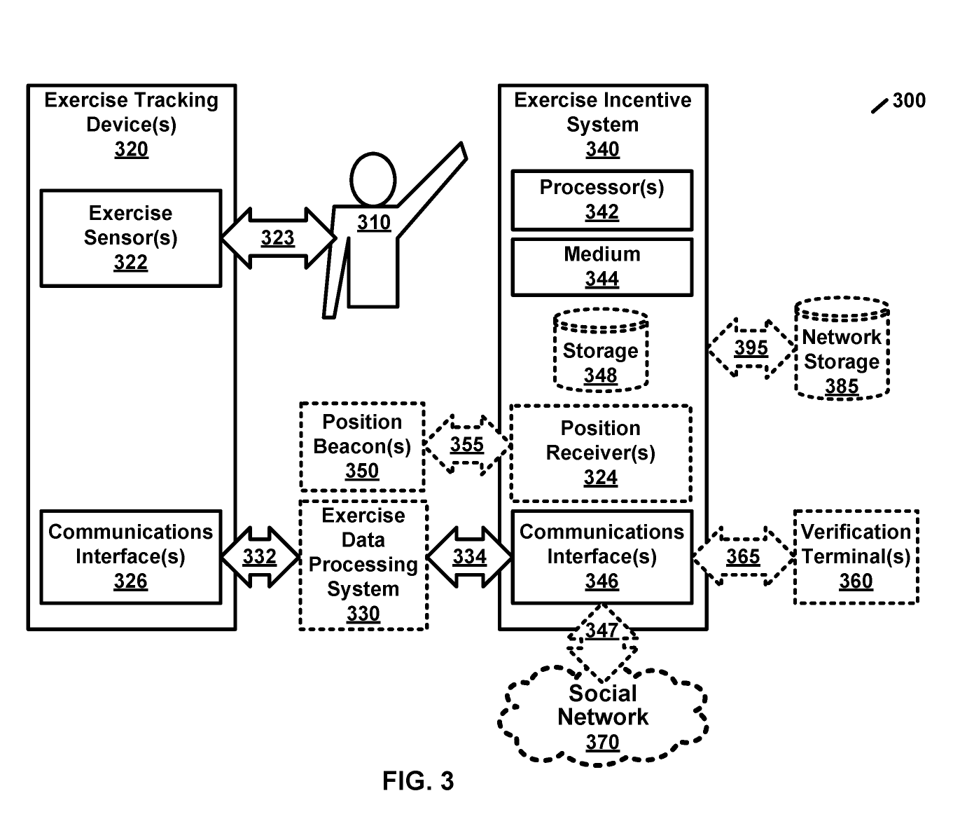

FIG. 3 is an example block diagram showing elements of an exercise incentive network 300 comprising an exerciser (e.g. a user) 310, at least one exercise tracking device 320, and an exercise incentive system 340. The exercise incentive system 340 may comprise at least one processor 342, at least one first communication interface 346, and a non-transitory machine readable medium 344. The at least one first communication interface 346 may be configured to receive exercise sensor data 334 wirelessly or via a wired connection. The exercise sensor data 334 may originate from at least one exercise tracking device 320.

According to some of the various embodiments, the at least one exercise tracking device 320 may comprise at least one exercise sensor 322 and at least one second communication interface 326. The at least one exercise sensor 322 may be coupled via link 323 to exerciser 310 over an interface. The at least one exercise tracking device 320 may comprise a fitness tracker, a biometric monitoring device, a heart rate monitor, a heart pulse transmitter, a position tracker, a movement tracker (e.g. motion sensor, step tracker, etc.), combinations thereof, and/or the like. According to some of the various embodiments, the at least one second communication interface 326 may be configured to transmit exercise sensor data 332 wirelessly or via a wired connection. The exercise sensor data 332 may be transmitted to an exercise incentive system 340. The exercise sensor data 332 may be determined from the at least one exercise sensor 322.

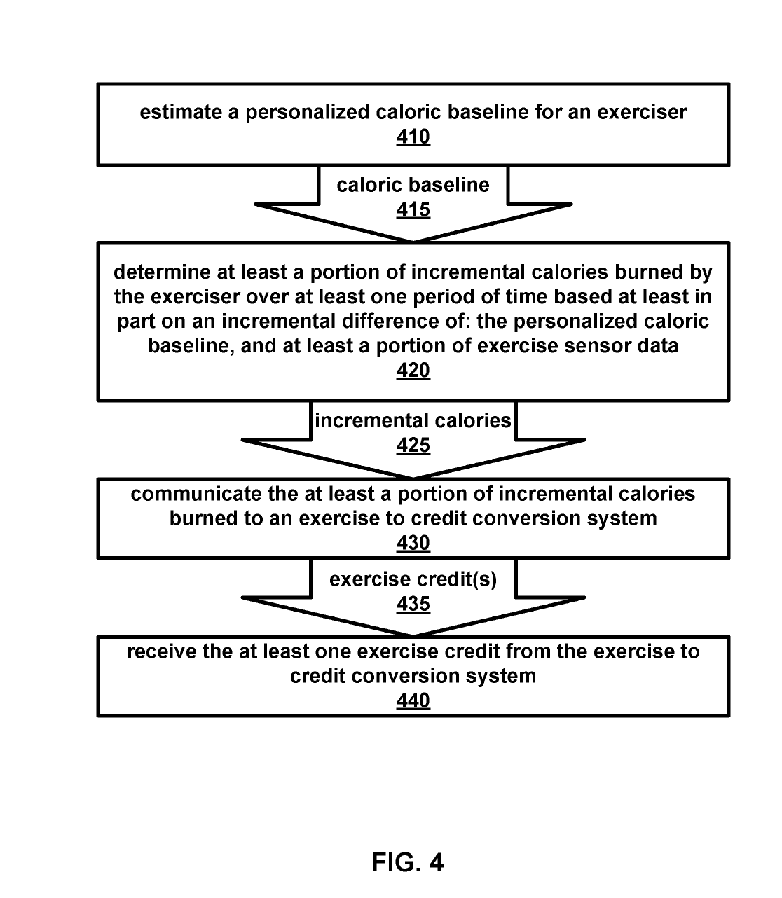

FIG. 4 is an example flow diagram of incremental calorie conversion as per an aspect of an embodiment. At 410, the non-transitory machine readable medium 344 may comprise instructions configured to cause the at least one processor 342 to estimate a personalized caloric baseline 415 for exerciser 310. The at least one processor 342 may employ exerciser specific data to estimate the personalized caloric baseline 415. The personalized caloric baseline 415 may be estimated over at least one period of time. The at least one period of time may comprise at least one second, at least one minute, at least one hour, at least one day, combinations thereof, and/or the like. The at least one period of time may be based on a period of exercise. The personalized caloric baseline 415 may be based at least in part on a Basal Metabolic Rate (BMR), a Resting Metabolic Rate (RMR), a Thermic Effect of Activity (TEA), a Thermic Effect of Feeding (TEF), an adaptive thermogenesis, a body weight of the exerciser, an age of the exerciser, a height of the exerciser, a gender of the exerciser, a lean body mass of the exerciser, combinations thereof, and/or the like. For example, BMR may be estimated by employing a Mifflin-St. Jeor Equation. The Mifflin-St. Jeor Equation is based in part on the gender of the exerciser. According to the Mifflin-St. Jeor Equation, BMR for men may be equal to 9.99×body weight (kg)+6.25×height (cm)−4.92×age (years)+5. BMR for women may be equal to 9.99×body weight (kg)+6.25×height (cm)−4.92×age (years)−161. Other examples of BMR estimation include the Harris-Benedict Equation and the Revised Harris-Benedict Equation. An example of RMR estimation is the Katch-McArdle Formula. According to the Katch-McArdle Formula, RMR may be equal to 570+41.6×LBM where LBM is lean body mass in kg. According to some of the various embodiments, the period of exercise may be identified over at least one time period. The period of exercise may be identified based on at least one threshold. The at least one threshold may comprise calories, steps, heart rate, distance, power output, combinations thereof, and/or the like.

At 420, the non-transitory machine readable medium 344 may comprise instructions configured to cause the at least one processor 342 to determine at least a portion of incremental calories 425 burned by the exerciser 310 over at least one period of time. The at least one period of time may comprise at least one second, at least one minute, at least one hour, combinations thereof, and/or the like. The at least one period of time may be based on a period of time in the exercise sensor data 334. The at least one period of time may comprise a duration of an exercise performed by the exerciser 310 and recorded by the at least one exercise tracking device 320. At 420, the at least a portion of incremental calories burned by the exerciser 310 may be determined based at least in part on an incremental difference of the personalized caloric baseline and at least a portion of the exercise sensor data 334. For example, a 30 year old female with weight of 175 lbs. and height of 5′10″ may have a BMR of 1,456 daily calories or 61 calories per hour 0.325 calories may be measured during a workout lasting one hour. Therefore, the incremental calories for the workout may comprise 325 minus 61 or 264 calories. In another example, the same female may measure a total calorie burn of 2,464 throughout the day. Therefore, incremental calories for the day may comprise 2,464 minus 1,456 or 1,008 calories. An advertiser may, for example, offer an exchange rate of 200 calories per $1 of eligible exercise credit. In this example, $2 of eligible credit may be earned for the workout and $5 of eligible credit may be earned for the daily activity.

At 430, the non-transitory machine readable medium 344 may comprise instructions configured to cause the at least one processor 342 to communicate the at least a portion of incremental calories burned 425 to an exercise to credit conversion system. The exercise to credit conversion system may be configured to convert the at least a portion of incremental calories burned 425 to at least one exercise credit 435. The at least one exercise credit 435 may comprise a digital currency, a currency, at least one coupon, at least one credit card, at least one debit card, at least one gift card, combinations thereof, and/or the like. Examples of digital currency include Bitcoin, Litecoin, Ripples, Stellars, combinations thereof, and/or the like. According to some of the various embodiments, the at least one credit card, at least one debit card, and/or at least one gift card may be physical, digital, temporary, configured for one-time use, combinations thereof, and/or the like. A physical credit card, debit card, and/or gift card may comprise a magnetic strip and/or a security chip. A digital credit card, debit card, and/or gift card may comprise a scanable barcode.

At 440, the non-transitory machine readable medium 344 may comprise instructions configured to cause the at least one processor 342 to receive the at least one exercise credit 435 from the exercise to credit conversion system.

According to some of the various embodiments, the at least one exercise credit 435 may be used towards an auction. The auction may comprise gift card(s), coupon(s), other item(s), combinations thereof, and/or the like. The auction may be sponsored by at least one advertiser. The at least one exercise credit 435 may be used towards a lottery. The lottery may be sponsored by at least one advertiser, at least one provider of an exercise incentive system, at least one provider of exercise credit, combinations thereof, and/or the like. The at least one exercise credit 435 may be configured to expire after a time duration. The time duration may be fixed or variable. For example, the time duration may comprise a day, a month, a quarter, a year, combinations thereof, and/or the like.

According to some of the various embodiments, the at least a portion of incremental calories burned 425 may be converted into at least one exercise credit 435 in at least one increment. The at least one increment may comprise a fixed increment, a variable increment, a pre-determined increment, combinations thereof, and/or the like. For example, digital gift cards may be available in increments of $5 US. In this example, additional incremental calories may be needed for exerciser 310 to reach the next available increment.

According to some of the various embodiments, the at least one second communication interface 326 may be integrated into one device (e.g. 320) comprising at least one exercise sensor 322. For example, a wearable activity tracker may comprise a position sensor and a Bluetooth Low Energy (BLE) transceiver. As a second example, a fitness tracker may comprise a step counter and a Universal Serial Bus (USB) interface. Alternatively, the at least one exercise sensor 322 may be integrated into a first device the at least one second communication interface 326 may be integrated into a second device. For example, a heart rate monitor may comprise a first device comprising a heart rate sensor (e.g. mounted on a chest strap) and a second device comprising an ANT+ transmitter. ANT+ may comprise a sub-system of the base ANT protocol (a proprietary wireless technology) designed and marketed by the ANT+ Alliance, a division of Dynastream Innovations Inc. ANT+ provides interoperability and open-access to data between manufacturers of, for example, smartphones, bike computers, diagnostics, power meters, heart rate monitors, remote control systems, combinations thereof, and/or the like. In this example, the first device may comprise a third communication interface configured to communicate with at least one fourth communication interface integrated into the second device.

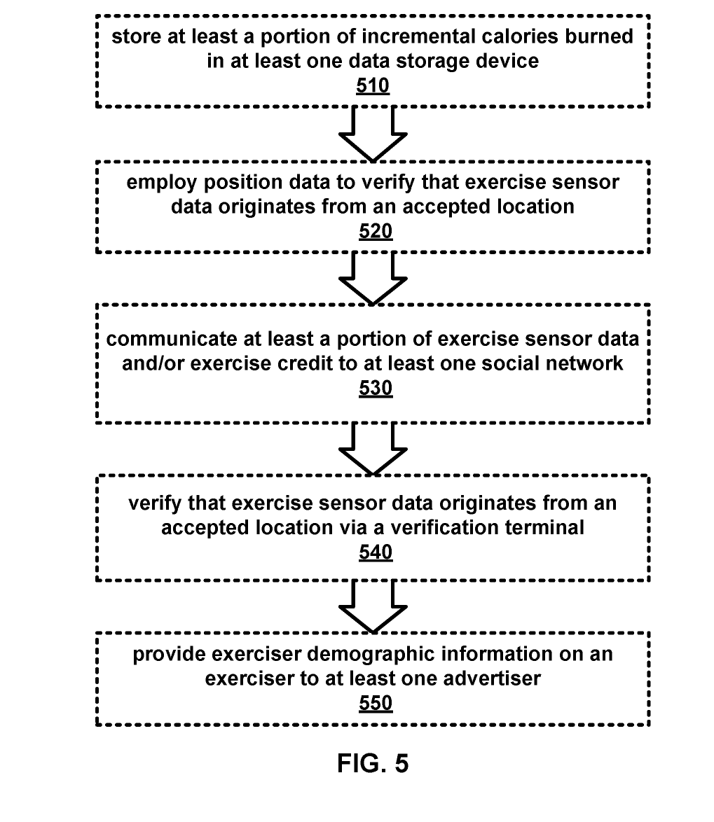

FIG. 5 is an example flow diagram of providing exerciser demographic information as per aspects of various embodiments. At 510, the non-transitory machine readable medium 344 may comprise instructions configured to cause the at least one processor 342 to store at least a portion of incremental calories burned (e.g. 425 and 395) in at least one data storage device (e.g. 348 or 395). The at least one data storage device (e.g. 348 or 395) may be integrated into the exercise incentive system 340, integrated into at least one verification terminal 360, located in the cloud, distributed across a plurality of computing devices, comprise a centralized database, combinations thereof, and/or the like.

At 520, the non-transitory machine readable medium 344 may comprise instructions configured to cause the at least one processor 342 to employ position data (e.g. 355) to verify that exercise sensor data originates from an accepted location. The exercise incentive system 340 may comprise at least one position receiver 324. The at least one position receiver 324 may be integrated into the at least one first communication interface 346. The at least one position receiver 324 may be configured to receive the position data 355. The position data 355 may be received from at least one position beacon 350. The at least one position beacon 350 may comprise at least one low-powered position transmitter. The at least one low-powered position transmitter may be configured to communicate position data 355. Examples of position beacons include iBeacon, Bluetooth beacon, a tablet with a known position broadcasted via Wi-Fi or NFC, a network node, combinations thereof, and/or the like. The position data 355 may comprise position and/or time data.

According to some of the various embodiments, the non-transitory machine readable medium 344 may comprise instructions configured to cause the at least one processor 342 to employ at least one exercise to credit exchange rate to convert the at least a portion of incremental calories burned 425 into the at least one exercise credit 435. The at least one exercise to credit exchange rate may be based at least in part on the exercise sensor data 334. The at least one exercise to credit exchange rate may differ for each type of burned calorie activity, the duration of each burned calorie activity, the location of each burned calorie activity, combinations thereof, and/or the like. The at least one exercise to credit exchange rate may be based at least in part on a cumulative total of incremental calories burned by exerciser 310. For example, the at least one exercise to credit exchange rate may be based on a fitness level of the exerciser 310.

At 530, the non-transitory machine readable medium 344 may comprise instructions configured to cause the at least one processor 342 to communicate at least a portion of the exercise sensor data 334 to at least one social network 370 via network connection 347. At 530, the non-transitory machine readable medium 344 may comprise instructions configured to cause the at least one processor 342 to communicate the at least one exercise credit 435 to at least one social network 370 via network connection 347.

According to some of the various embodiments, the exercise sensor data 334 may be the same as exercise sensor data 332. For example, at least one first communication interface 346 may be configured to communicate directly with at least one second communication interface 326. According to at least one alternative, the at least one first communication interface 346 may be configured to receive the exercise sensor data 334 originating from the at least one exercise tracking device 320 through at least one exercise data processing system 130. The at least one exercise data processing system 130 may reside in the non-transitory machine readable medium 344, in a machine readable medium associated with a distinct mobile device, in a machine readable medium associated with a distinct computing device, in a machine readable medium accessible over a network, combinations thereof, and/or the like. Communication with the at least one exercise data processing system 130 may employ at least one Application Programming Interface (API). At least one of the at least one API may comprise, for example, an API to a third party application such as, for example, Apple Health (e.g. via Healthkit), Google Fit, Microsoft Health Vault, MyFitnessPal, Strava, combinations thereof, and/or the like.

According to some of the various embodiments, the non-transitory machine readable medium 344 may comprise instructions configured to cause the at least one processor 342 to employ at least one verification terminal 360. At 540, the at least one verification terminal 360 may be employed to verify that exercise sensor data (e.g. 332, 334) originates from an accepted location. An accepted location may comprise, for example, a gym, an athletic facility, a health club, a start line, a finish line, a transition area in a triathlon, a piece of exercise equipment, combinations thereof, and/or the like.

According to some of the various embodiments, the at least one verification terminal 360 may comprise at least one second processor, at least one third communication interface, and a second non-transitory machine readable medium. The at least one verification terminal 360 may comprise a computing device, a computer server, a mobile device (e.g. a smartphone or tablet), combinations thereof, and/or the like. The at least one verification terminal 360 may be associated with a kiosk. The at least one third communication interface may be configured to receive exercise sensor data 332 originating from at least one exercise tracking device 320. Alternatively, the at least one third communication interface may be configured to receive exercise sensor data 334 from at least one exercise data processing system 130. The at least one third communication interface may be configured to receive exercise sensor data 334 wirelessly or via a wired connection. The second non-transitory machine readable medium may comprise instructions configured to cause the at least one second processor to verify that exercise sensor data (e.g. 332, 334) originates from an accepted location. The at least one verification terminal 360 may be configured to return verified exercise sensor data 365. The instructions may be configured to cause the at least one second processor to receive the exercise sensor data 334 from at least one exercise data processing system 130. The instructions may be configured to cause the at least one second processor to associate exerciser 310 with the exercise sensor data 334. The instructions may be configured to cause the at least one second processor to store the exercise sensor data (e.g. 334, 365) in at least one data storage device (e.g. 348).

At 550, the non-transitory machine readable medium 344 may comprise instructions configured to cause the at least one processor 342 to provide exerciser demographic information on exerciser 310 to at least one advertiser through at least one network interface (e.g. 346). The exerciser demographic information may comprise the exercise sensor data (e.g. 332, 334). The exerciser demographic information may comprise the at least a portion of incremental calories burned 425. At least a portion of the exerciser demographic information may be verified exerciser demographic information (e.g. 365). Verified exerciser demographic data (e.g. 365) may be verified through employment of, for example, a device such as verification terminal 360. Other mechanisms to verify data may be employed, such as for example, verification by a staff member, an attendant, a coach, a personal trainer, and/or the like. The exerciser demographic information may comprise a fitness level for exerciser 310. The fitness level may be based at least in part on a cumulative total of incremental calories burned by the exerciser 310. The exerciser demographic information may comprise at least one location associated with the exerciser 310. The at least one advertiser may comprise a retailer, a merchant, a brand, combinations thereof, and/or the like.

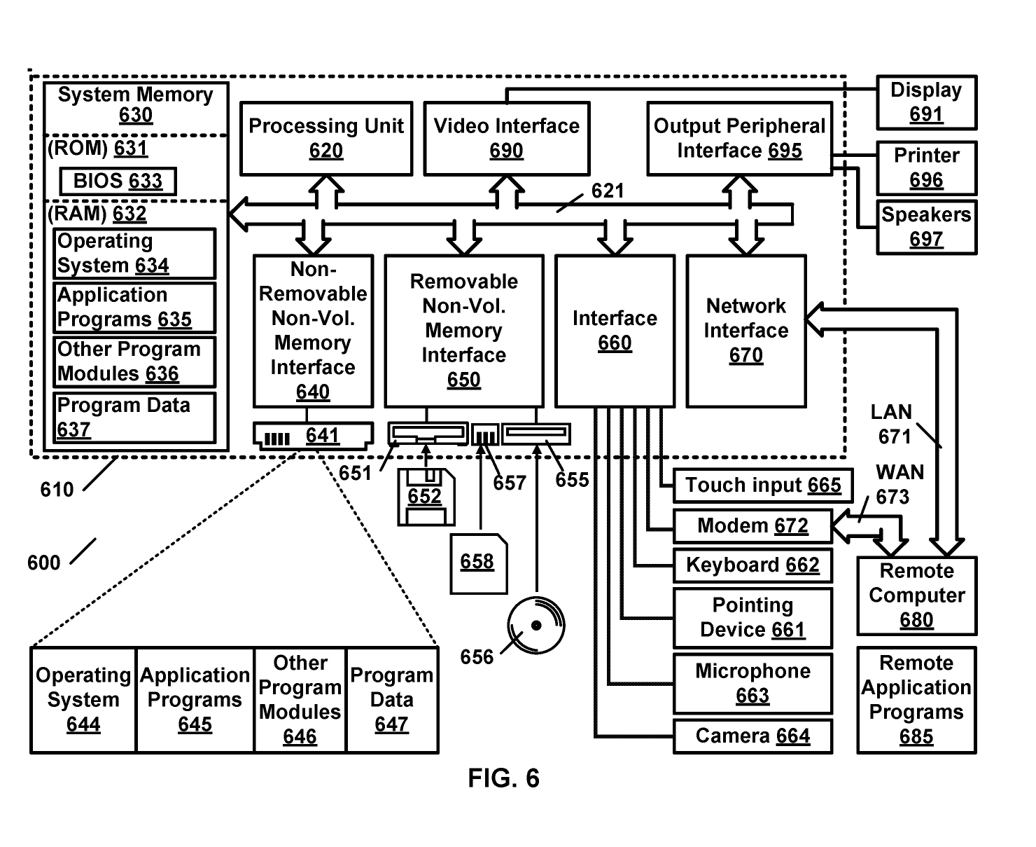

FIG. 6 illustrates an example system for implementing at least some embodiments and comprises a general-purpose computing device in the form of a computing device 610. Components of computing device 610 may include, but are not limited to, a processing unit 620, a system memory 630, and a system bus 681 that couples various system components including the system memory to the processing unit 620.

Computing device 610 may comprise a variety of computer readable media. Computer readable media may comprise available media accessible by computing device 610 and may comprise both volatile and nonvolatile media, removable and non-removable media. By way of example, and not limitation, computer readable media may comprise computer storage media and communication media. Computer storage media may comprise both volatile and nonvolatile, removable and non-removable media implemented by way of a method or technology for storage of information such as computer readable instructions, data structures, program modules or other data. Computer storage media may comprise, but is not limited to, random access memory (RAM), read-only memory (ROM), electrically erasable programmable read-only memory (EEPROM), flash memory or other memory technology, compact disc read-only memory (CD-ROM), digital versatile disks (DVD) or other optical disk storage, magnetic cassettes, magnetic tape, magnetic disk storage or other magnetic storage devices, or other medium which can be used to store the desired information and which can be accessed by computer 610. Communication media may embody computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media may comprise wired media such as a wired network or direct-wired connection, and wireless media such as acoustic, radio frequency (RF), infrared and other wireless media. Combinations of the above should also be included within the scope of computer readable media.

The system memory 630 may comprise computer storage media in the form of volatile and/or nonvolatile memory such as ROM 631 and RAM 632. A basic input/output system 633 (BIOS), containing the basic routines that help to transfer information between elements within computer 610, such as during start-up, may be stored in ROM 631. RAM 632 may comprise data and/or program modules that are accessible to and/or presently being operated on by processing unit 620. By way of example, and not limitation, FIG. 6 illustrates operating system 634, application programs 635, other program modules 636, and program data 637.

Computing device 610 may also comprise other removable/non-removable volatile/nonvolatile computer storage media. By way of example only, FIG. 6 illustrates a hard disk drive 641 that may read from or write to non-removable, nonvolatile magnetic media, a magnetic disk drive 651 that may read from or write to a removable, nonvolatile magnetic disk 652, a flash drive reader 657 that may read flash drive 658, and an optical disk drive 655 that may read from or write to a removable, nonvolatile optical disk 656 such as a CD ROM or other optical media. Other removable/non-removable, volatile/nonvolatile computer storage media that may be used in the operating environment include, but are not limited to, magnetic tape cassettes, flash memory cards, digital versatile disks, digital video tape, solid state RAM, solid state ROM, and the like. The hard disk drive 641 may be connected to system bus 681 through a non-removable memory interface such as interface 640. Magnetic disk drive 651 and optical disk drive 655 may be connected to the system bus 681 by a removable memory interface, such as interface 650.

The drives and their associated computer storage media discussed above and illustrated in FIG. 6, provide storage of computer readable instructions, data structures, program modules and other data for the computer 610. In FIG. 6, for example, hard disk drive 641 is illustrated as storing operating system 644, application programs 645, program data 647, and other program modules 646. Additionally, for example, non-volatile memory may comprise instructions for presenting images on a display 691 of computing device 600 and/or the like. Similarly, non-volatile memory may comprise instructions for causing the presentation of images on the display of a remote computing device 680 and/or the like. Display 691 and touch input 665 may be integrated into the same device.

A user may enter commands and information into computing device 610 through input devices such as a touch input device 665, a keyboard 662, a microphone 663, a camera 664, and a pointing device 661, such as a mouse, trackball or touch pad. These and other input devices may be connected to the processing unit 620 through a user input interface 660 that is coupled to system bus 621, but may be connected by other interface and bus structures, such as a parallel port, game port or a universal serial bus (USB). A display 691 or other type of display device may be connected to the system bus 681 via an interface, such as a video interface 690. Other devices, such as, for example, speakers 697 and printer 696 may be connected to the system via peripheral interface 695.

The computing device 610 may be operated in a networked environment using logical connections to one or more remote computers, such as a remote computer 680. The remote computer 680 may be a personal computer, a hand-held device, a server, a router, a network PC, a peer device or other common network node, and typically includes many or all of the elements described above relative to the computing device 610. The logical connections depicted in FIG. 6 include a local area network (LAN) 671 and a wide area network (WAN) 673, but may also include other networks. Such networking environments are commonplace in offices, enterprise-wide computer networks, intranets and the Internet.

When used in a LAN networking environment, the computing device 610 is connected to the LAN 671 through a network interface or adapter 670. When used in a WAN networking environment, the computing device 610 may comprise a modem 672 or other means for establishing communications over the WAN 673, such as the Internet. The modem 672, which may be internal or external, and may be connected to the system bus 681 via interface 660, or other appropriate mechanism. Modem 672 may be wired or wireless. Examples of wireless devices may comprise, but are not limited to: Wi-Fi and BluetoothTM. In a networked environment, program modules depicted relative to the computing device 610, or portions thereof, may be stored in a remote memory storage device. By way of example, and not limitation, FIG. 6 illustrates remote application programs 685 as residing on remote computer 680. It will be appreciated that the network connections shown are presented as examples only and other means of establishing a communications link between the computers may be used.

Although the subject matter has been described in language specific to system features and/or instructions, it is to be understood that the subject matter defined in the appended claims is not necessarily limited to the specific features or instructions described above. Rather, the specific features and instructions described above are disclosed as example forms of implementing the claims.

In this specification, “a” and “an” and similar phrases are to be interpreted as “at least one” and “one or more.” References to “an” embodiment in this disclosure are not necessarily to the same embodiment.

Many of the elements described in the disclosed embodiments may be implemented as modules. A module is defined here as an isolatable element that performs a defined function and has a defined interface to other elements. The modules described in this disclosure may be implemented in hardware, a combination of hardware and software, firmware, wetware (i.e. hardware with a biological element) or a combination thereof, all of which may be behaviorally equivalent. For example, modules may be implemented using computer hardware in combination with software routine(s) written in a computer language (Java, HTML, XML, PHP, Python, ActionScript, JavaScript, Ruby, Prolog, SQL, VBScript, Visual Basic, Perl, C, C++, Objective-C or the like). Additionally, it may be possible to implement modules using physical hardware that incorporates discrete or programmable analog, digital and/or quantum hardware. Examples of programmable hardware include: computers, microcontrollers, microprocessors, application-specific integrated circuits (ASICs), field programmable gate arrays (FPGAs), and complex programmable logic devices (CPLDs). Computers, microcontrollers and microprocessors are programmed using languages such as assembly, C, C++ or the like. FPGAs, ASICs and CPLDs are often programmed using hardware description languages (HDL) such as VHSIC hardware description language (VHDL) or Verilog that configure connections between internal hardware modules with lesser functionality on a programmable device. Finally, it needs to be emphasized that the above mentioned technologies may be used in combination to achieve the result of a functional module.

Some embodiments may employ processing hardware. Processing hardware may include one or more processors, computer equipment, embedded system, machines and/or the like. The processing hardware may be configured to execute instructions. The instructions may be stored on a machine-readable medium. According to some embodiments, the machine-readable medium (e.g. automated data medium) may be a medium configured to store data in a machine-readable format that may be accessed by an automated sensing device. Examples of machine-readable media include: magnetic disks, cards, tapes, and drums, flash memory, memory cards, electrically erasable programmable read-only memory (EEPROM), solid state drives, optical disks, barcodes, magnetic ink characters, and/or the like.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above described exemplary embodiments. In particular, it should be noted that, for example purposes, systems have been described as including at least one processor. However, one skilled in the art will recognize that the at least one processor may be distributed among at least one server and at least one client device. The at least one server and at least one client device may vary from traditional server/device relationships over a network such as the internet. For example, a server may be collective based: portable equipment, broadcast equipment, virtual, application(s) distributed over a broad combination of computing sources, part of a cloud, and/or the like. Similarly, for example, a client device may be a user based client, portable equipment, mobile equipment, broadcast equipment, virtual, application(s) distributed over a broad combination of computing sources, part of a cloud, and/or the like. Additionally, it should be noted that, for example purposes, several of the various embodiments were described with instructions. However, one skilled in the art will recognize that many various languages and frameworks may be employed to build and use embodiments of the present invention. For example, languages/frameworks may be based upon Java, HTML, XML, PHP, Python, ActionScript, JavaScript, Ruby, Prolog, SQL, VBScript, Visual Basic, Perl, C, C++, Objective-C combinations thereof, and/or the like.

In addition, it should be understood that any figures that highlight any functionality and/or advantages, are presented for example purposes only. The disclosed architecture is sufficiently flexible and configurable, such that it may be utilized in ways other than that shown. For example, the steps listed in any flowchart may be re-ordered or only optionally used in some embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112. Claims that do not expressly include the phrase “means for” or “step for” are not to be interpreted under 35 U.S.C. 112.

What is claimed is:

1. An exercise to credit conversion system coupled to a global computer network with access to at least one storage device and programmed to perform a method of converting exercise data into at least one exercise credit, the method performed by the exercise to credit conversion system comprising:
a. receiving exercise to credit exchange information from at least one advertiser computer system;
b. receiving exerciser attribute information from an exercise incentive system;
c. receiving exercise data from at least one of the following:
i. the exercise incentive system; and
ii. at least one exercise data processing system;
d. storing the exercise data as stored exercise data in at least one of the at least one storage device;
e. creating exerciser demographic information based at least in part on at least one of the following:
i. exerciser attribute information; and
ii. stored exercise data;
f. communicating the exerciser demographic information to the at least one advertiser computer system;
g. creating at least one eligible exercise credit based at least in part on the exercise to credit exchange information and the stored exercise data; and
h. communicating the at least one eligible exercise credit to the exercise incentive system.

2. The system according to claim 1, wherein the exerciser attribute information comprises at least one of the following:
a. age;
b. height;
c. weight; and
d. gender.

3. The system according to claim 1, wherein the exerciser attribute information comprises at least one location.

4. The system according to claim 1, wherein at least some of the exercise data is derived from exercise sensor data electronically communicated from at least one of the following:
a. a fitness tracker;
b. a biometric monitoring device;
c. a heart rate monitor;
d. a heart pulse transmitter;
e. a position tracker; and
f. a movement tracker.

5. The system according to claim 1, wherein the at least one eligible exercise credit comprises at least one of the following:
a. a digital currency;
b. a currency;
c. at least one electronic coupon;
d. at least one credit card code;
e. at least one debit card code; and
f. at least one gift card code.

6. The system according to claim 1, wherein the exerciser demographic information further comprises a fitness level for an exerciser, the fitness level based at least in part on the stored exercise data associated with the exerciser, the stored exercise data comprising data associated with a plurality of exercises.

7. The system according to claim 1, the method performed by the exercise to credit conversion system further comprising receiving a selection of at least one of the at least one eligible exercise credit from the exercise incentive system.

8. The system according to claim 1, the method performed by the exercise to credit conversion system further comprising:
a. creating at least one exercise credit selection report based at least in part on at least one selection of at least one of the at least one eligible exercise credit communicated from the exercise incentive system; and
b. communicating the at least one exercise credit selection report to at least one of the at least one advertiser computer system.

9. The system according to claim 1, the method performed by the exercise to credit conversion system further comprising communicating at least one exercise effort report to the exercise incentive system.

10. The system according to claim 1, wherein at least one of the at least one advertiser computer system is configured to communicate at least one exerciser targeted advertisement to the exercise incentive system, the at least one exerciser targeted advertisement based, at least in part, on the exerciser demographic information.

11. An exercise incentive system comprising:
a. at least one processor;
b. at least one first communication interface configured to receive exercise sensor data originating from at least one exercise tracking device, the at least one exercise tracking device comprising:
i. at least one exercise sensor; and
ii. at least one second communication interface configured to transmit the exercise sensor data determined from the at least one exercise sensor; and
c. a non-transitory machine readable medium comprising instructions configured to cause the at least one processor to:
i. estimate a personalized caloric baseline for an exerciser;
ii. determine at least a portion of incremental calories burned by the exerciser over at least one period of time based at least in part on an incremental difference of:
(1) the personalized caloric baseline; and
(2) at least a portion of the exercise sensor data;
iii. communicate the at least a portion of incremental calories burned to an exercise to credit conversion system configured to convert the at least a portion of incremental calories burned to at least one exercise credit; and
iv. receive the at least one exercise credit from the exercise to credit conversion system.

12. The system according to claim 11, wherein the personalized caloric baseline is based at least in part on at least one of the following;
a. a Basal Metabolic Rate (BMR);
b. a Resting Metabolic Rate (RMR);
c. a Thermic Effect of Activity (TEA);
d. a Thermic Effect of Feeding (TEF);
e. an adaptive thermogenesis;
f. a body weight of the exerciser;
g. an age of the exerciser;
h. a height of the exerciser;
i. a gender of the exerciser; and
j. a lean body mass of the exerciser.

13. The system according to claim 11, wherein the at least one period of time is based on a period of time in the exercise sensor data.

14. The system according to claim 11, wherein the at least one period of time comprises a duration of an exercise performed by the exerciser and recorded by the at least one exercise tracking device.

15. The system according to claim 11, wherein the instructions are further configured to cause the at least one processor to employ position data to verify that exercise sensor data originates from an accepted location.

16. The system according to claim 11, further comprising at least one position receiver configured to receive position data from at least one position beacon.

17. The system according to claim 11, wherein the at least one exercise credit is based on at least one of: an exercise to credit exchange rate and the at least a portion of incremental calories burned.

18. The system according to claim 17, wherein the exercise to credit exchange rate is based at least in part on a cumulative total of incremental calories burned by the exerciser.

19. The system according to claim 11, wherein the at least one first communication interface is configured to receive the exercise sensor data originating from the at least one exercise tracking device through at least one exercise data processing system.

20. The system according to claim 11, wherein the instructions are further configured to cause the at least one processor to employ at least one verification terminal to verify that exercise sensor data originates from an accepted location.
